(19) United States
(12) Patent Application Publication
(10) Pub. No.: US 2026/0020994 A1
(43) Pub. Date: Jan. 22, 2026

(54) NONWOVEN AND ABSORBENT ARTICLES HAVING THE SAME

(71) Applicant: THE PROCTER & GAMBLE COMPANY, Cincinnati, OH (US)

(72) Inventors: Xiaoxin LIU, Beijing (CN); Xiaohui DONG, Beijing (CN); Xue WANG, Beijing (CN); Matteo GHIOLDI, Frankfurt (DE); Gerard Alain VIENS, Cincinnati, OH (US)

(21) Appl. No.: 19/269,110

(22) Filed: Jul. 15, 2025

(30) Foreign Application Priority Data
Jul. 16, 2024 (WO) PCT/CN2024/105604

(51) Int. Cl. A61F 13/53; A61F 13/511

(52) U.S. Cl. CPC A61F 13/53; A61F 13/51121

(57) ABSTRACT

The present disclosure relates to a nonwoven including a first layer and a second layer. The first layer has first fibers and a first mean pore size. The first fibers have a first fiber fineness. The second layer includes second fibers and a second mean pore size. The second fibers have a second fiber fineness, and the second fiber fineness is equal to or greater than the first fiber fineness, and the second mean pore size is smaller than the first mean pore size. The first fibers may include a polymer and the polymer may include polytrimethylene terephthalate, polybutylene terephthalate and a combination thereof. The second fiber may include a thermoplastic fiber. The nonwoven may be included in an absorbent article. At least one of the topsheet and the fluid management layer of the absorbent article may include the nonwoven.

### CROSS REFERENCE TO RELATED APPLICATION

This application claims the benefit of PCT/CN2024/105604, filed Jul. 16, 2024, the entire disclosure of which is fully incorporated by reference herein.

### FIELD OF THE INVENTION

The present invention relates to nonwoven, and an absorbent article comprising the nonwoven.

### BACKGROUND

Nonwovens including synthetic fibers formed from thermoplastic resin are widely used as sheets constituting absorbent articles such as sanitary napkins, infant disposable diapers, personal care disposable diapers, and the like.

These absorbent articles comprise several layers providing different functions. A liquid permeable topsheet is disposed closest to the wearer's skin and should be capable of quickly absorbing the excreted fluid. A backsheet is disposed on the opposed, garment-facing side of the article. Some absorbent articles in the market further comprises a nonwoven outermost layer forming at least part of a garment-facing surface of an absorbent article. Other components of absorbent articles are well known, and include in particular an absorbent core disposed between the topsheet and the backsheet to absorb and retain the excreted fluids.

It is desirable in an absorbent article that the body fluid discharged on the topsheet rapidly transfer from a top surface of the topsheet towards the bottom of the topsheet which usually keep in close contact with an adjacent layer such as a fluid management layer and an absorbent core of the absorbent article, so that the body fluid rapidly transfers from the topsheet into the adjacent layer and eventually an absorbent core without giving a wearer uncomfortable feeling of wetness.

So as not to prevent liquid transfer from a topsheet to the next layer and eventually an absorbent core and minimize an amount of body fluid remaining on the topsheet, absorbent articles having a capillary gradient is desirable as it can provide better fluid drainage from a wearer-facing surface of the topsheet to an adjacent layer which can improve dryness and/or cleanness of an absorbent article. Capillary gradient in an absorbent article may be generated by creating a pore size gradient from the top to bottom of the absorbent article in such a way that a pore size is greater in an upper layer than in a lower layer so that the lower layer has high capillary suction power than the upper layer. One approach to form pore size gradient is to design nonwoven having an upper part constituted with higher denier fibers and a lower part constituted with smaller denier fibers. However, employment of a high denier fibers on an upper side or upper layer compromises smoothness and gentleness of the upper side or upper layer.

Therefore, there is a continuous need for a nonwoven having a pore size gradient without compromising surface smoothness.

There is a continuous need for an absorbent article that provides a high fluid absorption speed and mitigated rewet and still can have a smooth surface facing wearer's skin.

### SUMMARY

The present invention provides a nonwoven comprising a first layer comprising first fibers and having a first mean pore size as measured by Pore Size Measurement, the first fiber having a first fiber fineness as measured by Fiber Decitex Measurement, a second layer comprising second fibers and having a second mean pore size as measured by Pore Size Measurement, the second fiber having a second fiber fineness as measured by Fiber Decitex Measurement, wherein the second fiber fineness is equal to or greater than the first fiber fineness, and wherein the second mean pore size is smaller than the first mean pore size.

The present invention also provides a nonwoven comprising a first layer comprising first fibers, the first fiber comprising a polymer selected from the group consisting of comprising PTT, PBT and a combination thereof, and a second layer comprising second fibers, the second fiber comprises a thermoplastic fiber.

The present invention also provides an absorbent article having a wearer-facing surface and a garment-facing surface, the absorbent article comprising a topsheet, a backsheet, an absorbent core disposed between the topsheet and the backsheet, and an optional fluid management layer disposed between the topsheet and the absorbent core, wherein at least one of the topsheet and the fluid management layer comprises the nonwoven of the present invention in such a way that the first layer towards the wearer-facing surface.

These and other features, aspects, and advantages of the present invention will become evident to those skilled in the art from a reading of the present disclosure.

### BRIEF DESCRIPTION OF THE DRAWINGS

FIG. 1A is a microscope image of a cross-section of a nonwoven of the present invention.

FIG. 1B is a microscope image of a cross-section of the nonwoven of FIG. 1A after reloft.

FIG. 2 is a microscope image of a cross-section of another nonwoven of the present invention.

FIG. 3 is a perspective view of an exemplary absorbent article.

FIG. 4 is a microscope image of a cross-section of a nonwoven illustrating nonwoven caliper measurement.

FIG. 5 is a microscope image of a cross-section of a conventional nonwoven.

FIG. 6 is a microscope image of a cross-section of another conventional nonwoven.

FIG. 7 is a microscope image of a partial cross-section of an absorbent article according to the present invention.

### DETAILED DESCRIPTION

All ranges are inclusive and combinable. The number of significant digits conveys neither limitations on the indicated amounts nor on the accuracy of the measurements. All numerical amounts are understood to be modified by the word “about” unless otherwise specifically indicated.

The term “absorbent articles”, as used herein, include disposable diapers, sanitary napkins, menstrual pants, panty liners, incontinence pads, interlabial pads, breast-milk pads, sweat sheets, animal-use excreta handling articles, animal-use diapers, and the like.

The term “component” of an absorbent article, as used herein, refers to an individual constituent of an absorbent article, such as a topsheet, a fluid management layer such as a fluid acquisition layer and a fluid distribution layer, absorbent core, or layers of absorbent cores, and a backsheet.

The “Z-direction” is orthogonal to both the longitudinal and transverse directions.

### Nonwoven

In one aspect, the nonwoven of the present disclosure has a pore size gradient in such a way that an upper part of the nonwoven has a bigger mean pore size than a lower part of the nonwoven and the upper part of the nonwoven is constituted with fibers having a fiber fineness equal to or smaller than fibers constituting the lower part of the nonwoven.

In another aspect, the present invention provides a nonwoven comprising a first layer comprising first fibers, the first fiber comprising a polymer selected from the group consisting of comprising PTT, PBT and a combination thereof, and a second layer comprising second fibers, the second fiber comprises a thermoplastic fiber.

The nonwoven of the present disclosure comprises at least two nonwoven webs or layers (herein after, “nonwoven layers”, collectively). The at least two nonwoven layers may be attached to one another by, for example, thermal bonding, adhesive bonding or a combination thereof. Alternatively, the at least two nonwoven layers may be integrated. The term “integrated” as used herein is used to describe fibers of a nonwoven material which have been intertwined, entangled, and/or pushed/pulled in a positive and/or negative Z-direction (direction of the thickness of the nonwoven material). Some exemplary processes for integrating fibers of a nonwoven web include spunlacing and needlepunching. In one embodiment, the nonwoven of the present invention is a carded air-through nonwoven.

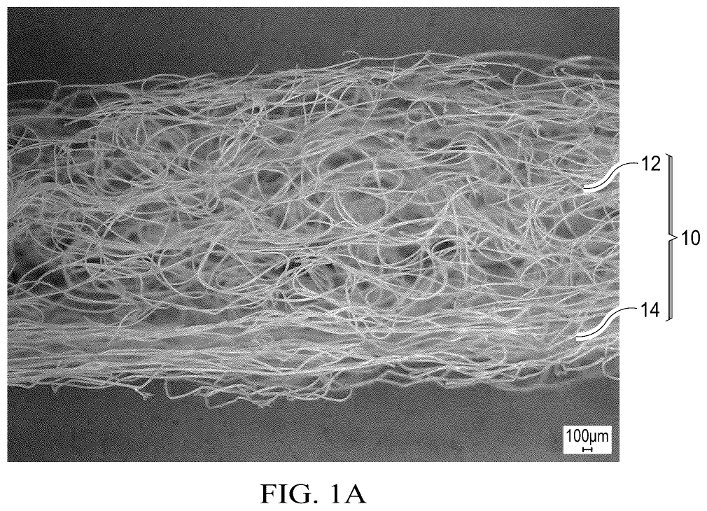

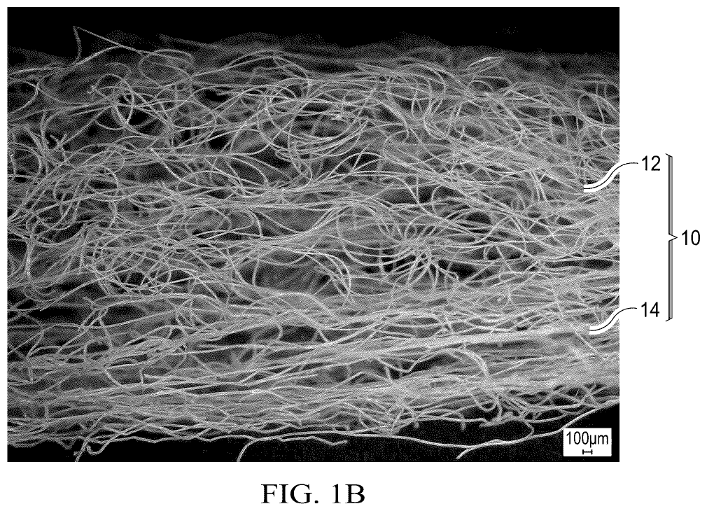

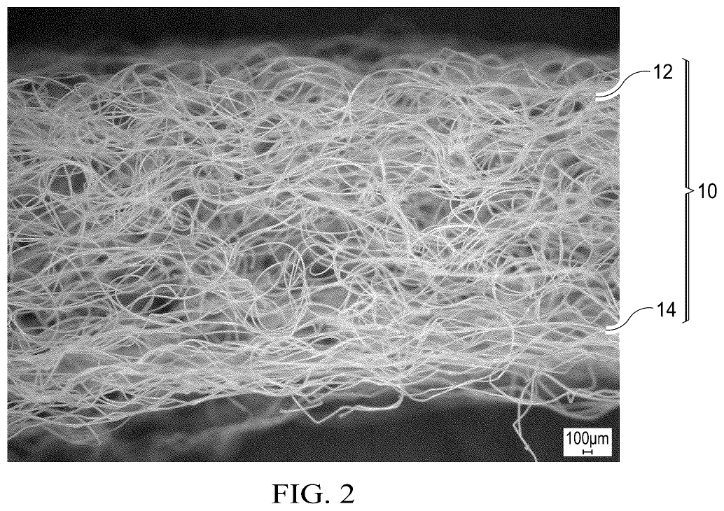

FIG. 1A is a microscope image of a cross-section of one nonwoven of the present invention, and FIG. 1B is a microscope images of the same nonwoven after relofted at 55° C. for 5 minutes. FIG. 2 is a microscope image of a cross-section of another nonwoven of the present invention.

Referring to FIGS. 1A and 2, a nonwoven 10 of the present invention comprises a first layer 12 comprising first fibers, and a second layer 14 comprising second fibers. The first layer has a first mean pore size, and the second layer has a second mean pore size which is smaller than the first mean pore size as measured according to Pore Size Measurement disclosed herein. The first fiber constituting the first layer has a first fiber fineness, and the second fiber constituting the second layer has a second fiber fineness which is equal to or greater than the first fiber fineness as measured according to Fiber Decitex Measurement disclosed herein.

The nonwoven configuration where the second layer of the nonwoven has a smaller mean pore size than the first layer of the nonwoven introduces a capillary gradient in the nonwoven. Due to the pore size gradient, the nonwoven can effectively transfer the body fluid from the top layer to the second layer which can improve dryness and cleanness of an absorbent article when the nonwoven is used as a component such as a topsheet or a fluid management layer of the absorbent article. According to the present invention such capillary gradient in the nonwoven can be created even with a first layer comprising first fibers having a fiber fineness equal to or smaller than a fiber fineness of the second fiber constituting the second layer, and the nonwoven still can provide a first layer surface with a desirable smoothness and soft comfortable feeling.

In addition, the nonwoven can provide a high caliper recovery factor when relofted even at a lower reloft temperature such as about 60° C. or about 55° C.

In some configurations, nonwovens are supplied on rolls and moved to an absorbent article manufacturing location. During the absorbent article assembly process, nonwovens are unwound from the rolls and supplied to an assembly line that converts the nonwoven of material into absorbent articles. In some instances, nonwovens may be relatively tightly wound on the rolls, and as such, the associated high winding pressures may compress nonwoven webs, resulting in a reduced caliper. Such compressed nonwoven webs when incorporated into an absorbent article may have a thin appearance that conveys a message of reduced softness to a consumer and/or may be aesthetically unpleasing. They may also negatively affect various performances of the nonwoven webs. To mitigate the problems associated with nonwoven compression, some manufacturers may apply heat to the nonwoven once unwound from the rolls. In turn, the application of heat to some types of nonwoven may increase the caliper or volume of the web materials, referred to herein as “relofting”.

The nonwoven of the present invention can provide a caliper recovery factor no less than about 5%, or no less than about 6% or no less than about 7% as measured according to Caliper Recovery Factor.

In some embodiments, the first layer 12 and the second layer 14 are bonded by pressure and/or thermo-bond between fibers in the first layer 12 and the second layer 14. The first layer 12 and the second layer 14 in the nonwoven 10 may be bonded to each other without using chemicals such as adhesive and latex. In the embodiments, at least part of fibers of the second layer 14 may extend into the first layer 12.

Fibers constituting the nonwoven of the present invention can be continuous, such as those produced by spunbonded methods, or cut to length, such as those typically utilized in a carded process. Fibers can be bicomponent, multiconstituent, shaped, crimped, or in any other formulation or configuration known in the art for nonwoven and fibers. In general, the fibers can be bondable, either by chemical bond (e.g., by latex or adhesive bonding), pressure bonding, or thermal bonding.

The nonwoven of the present invention may comprise deformations such as apertures, protrusions, recesses, and any combinations thereof.

A basis weight of the nonwoven may be appropriately selected depending on the nonwoven application. A basis weight of the first layer and the second layer, respectively, may be from about 5 g/m² to about 80 g/m², from about 10 g/m² to about 70 g/m², or from about 20 g/m² to about 50 g/m².

A ratio of a basis weight of the first layer to the second layer may be from about 80/20 to about 20/80, or from about 60/40 to about 50/50. If the basis weight of the first layer is too small and/or the ratio of the basis weight of the first layer to the basis weight of the second layer is too low, then the bulkiness and cushiony feel, and smoothness in the surface of the nonwoven may decline, the caliper recovery of the nonwoven may also decline and the nonwoven may not provide sufficient void volume. If the basis weight of the first layer is too large and/or the ratio of the basis weight of the first layer to the basis weight of the first layer is too high, uniformity of the nonwoven may be deteriorated, and/or fail to provide enough capillary capability to drain fluid from the first layer to second layer.

When the nonwoven of the present invention is used as a topsheet of an absorbent article, the basis weight of the nonwoven may be from about 15 g/m² to about 100 g/m², or about 25 g/m² to about 65 g/m², or about 30 g/m² to about 60 g/m^{2.} When the nonwoven of the present invention is used as a fluid management layer of an absorbent article, the basis weight of the nonwoven may be in the range of from about 30 g/m² to about 150 g/m², or from about 40 g/m² to about 100 g/m².

In one embodiment, the first layer is less hydrophilic than the second layer in the nonwoven of the present invention. In another embodiment, the first layer is more hydrophilic than the second layer in the nonwoven of the present invention.

A nonwoven disclosed herein can be used in a variety of disposable absorbent articles, but is particularly useful in diapers, feminine hygiene products and incontinence products such as sanitary napkins and incontinence pads. The nonwoven of the present disclosure may be particularly effective as a topsheet and a fluid management layer in the above absorbent articles. It may also be effective an outer most layer in absorbent articles.

### First Layer

The first layer in the nonwoven of the present invention comprises first fibers having a first fiber fineness, and has a first mean pore size.

The first fiber may comprise a fiber recovery no greater than about 6%, or no greater than about 5%, or no greater than about 4.5% as measured according to Fiber Property Test. The first fiber may comprise a fiber elasticity rate no lower than about 80%, or no lower than about 85%, or no lower than about 88% as measured according to Fiber Property Test.

In some examples, the first fiber comprising a polymer selected from the group consisting of comprising polytrimethylene terephthalate (“PTT”), polybutylene terephthalate (“PBT”) and a combination thereof. Without bound to a particular theory, unique molecular structure of resins such as having additional methylene groups on the fiber structure may enable the fibers to form z-shaped spiral molecular chains and maintain curl retention even under a compressed condition during winded storage on a roll which can result in a relatively big pore size despite smaller dtex. Thanks to the retention of curly structure, the first fiber may have a higher elasticity and an elastic recovery.

In some examples, the first fiber is a monocomponent fiber such as PTT and PBT. In some examples, the first fiber is a fiber selected from the group consisting of PTT, PBT, a composite fiber comprising PTT or PBT, and any combination thereof. The composite fiber comprising PTT or PBT may be composite fibers further comprising polyolefin polymer such as polyethylene (“PE”) and polypropylene (“PP”). The composite fiber may be a core/sheath composite fiber where a core component comprises PTT or PBT. Use of a composite fiber as the first fiber enables the first layer to have good integrity by having adhesions among fibers.

The first fiber forming the first layer have a fiber thickness in the range from about 0.8 denier to about 6 denier, or from about 1.0 denier to about 6 denier, or from about 1.5 denier to about 5 denier, or from about 2 denier to about 5 denier. The first fiber may have a fiber length less than about 100 mm. The first fiber may be a thermoplastic fiber.

The first layer may further comprise optional third fibers. When the first layer comprises third fibers, the third fiber may comprise thermoplastic fiber. The third fiber may be a multicomponent composite fiber comprising polyolefin polymer such as PE/PET. The third fiber may be a monocomponent fiber such as polyester terephthalate (PET). The composite fiber includes core/sheath composite fibers and side-by-side composite fibers.

In some embodiment, the first layer is a carded air-through nonwoven.

### Second Layer

The second layer comprises second fibers having a second fiber fineness, and has a second mean pore size. The second fiber may comprise a thermoplastic fiber including a composite fiber comprising polyolefin polymer such as polyethylene (“PE”) and polypropylene (“PP”). The composite fiber includes core/sheath composite fibers, and side-by-side composite fibers comprising polyolefin polymer such as PE and PP. Use of a composite fiber as the second fiber enables the second layer to have good integrity by having adhesions among fibers.

Thermoplastic composite fibers suitable for the present invention may have two-dimensional crimps and/or three-dimensional crimps. Herein, the term “two-dimensional crimp” can be understood mechanical crimping in which the peaks of the crimped fiber are sharply angled. Three-dimensional crimp may refer to crimp where the peaks are curved (wave shaped crimping) or spiral (spiral shaped crimping), crimp where both wave shaped crimping and spiral shaped crimping exist, or crimp where both mechanical crimp and at least one of wave and spiral shape crimps exist. In one embodiment, the core/sheath composite fiber has two-dimensional crimps which is cost-effective compared to a composite fiber having three-dimensional crimps.

Thermoplastic composite fibers suitable for the present invention may be concentric or eccentric.

The core component comprises at least one resin, thermoplastic resin preferably. Resin for the core component preferably includes a polyolefin-based resin such as polypropylene, polymethylpentene, and the like; polyester resins such as polyethylene terephthalate, polybutylene terephthalate, polytrimethylene terephthalate, polyethylene naphthalate, polylactic acid, and copolymers thereof; polyamide-based resins such as nylon 66, nylon 12, nylon 6, and the like; acrylic resin; engineering plastics such as polycarbonate, polyacetal, polystyrene, cyclic polyolefin, and the like; mixtures thereof. For the perspectives of the uniformity of the nonwoven and nonwoven productivity, polyolefin resin, polyester and polyamide-based resin are more preferable. Examples of the polyester include polymers and copolymers such as polyethylene terephthalate, polybutylene terephthalate, polytrimethylene terephthalate, polyethylene naphthalate, polylactic acid. The core component may be selected from the group consisting of polyethylene terephthalate, polybutylene terephthalate, polypropylene, nylon, polyamide, and combinations thereof. Polyethylene terephthalate and polybutylene terephthalate are preferred, and polyethylene terephthalate are more preferred. Alternatively, the core component may comprise only polyester as a polymer component.

The core component may comprise additives other than resin, such as anti-static agents, pigments, matting agents, thermal stabilizers, light stabilizers, flame retardants, antimicrobial agents, lubricants, plasticizers, softeners, antioxidants, ultraviolet absorbers, crystal nucleating agents, and the like. These additives may be included in the core component at an amount that is not more than about 10 mass % of the core component.

The sheath component of the core/sheath composite fiber comprises a thermoplastic resin having a melting point that is at least about 20° C. lower than a melting point of the resin in the core component of the core/sheath composite fiber. The thermoplastic resin suitable for the sheath component may include resins described with respect to the core component above.

In some embodiments, the second fiber is different from the first fiber at least in polymers constituting the fibers. The second fiber may not comprise either PTT or PBT.

The second fiber has a second fiber fineness equal to or greater than the fiber fineness of the first fiber constituting the first layer. The second fiber may have a fiber thickness in the range from about 1.5 denier to about 6 denier, or from about 2 denier to about 6 denier, or from 3 denier to about 5 denier. The second fiber may have a fiber length less than about 100 mm.

The second layer may be hydrophilic.

In some embodiment, the second layer is a carded air-through nonwoven.

### Nonwoven Manufacturing Process

The nonwoven of the present invention may be manufactured by any suitable methods known in the art such as meltblowing, spunbonding, hydroentangling, airlaid, wetlaid, and carded thermal bonding such as carded air-through bonding process.

The nonwoven may be manufactured via a process comprising the steps of: (a) forming a first fibrous web comprising first fibers, and forming a second fibrous web comprising second fibers; (b) forming a composite fibrous web by overlaying the first fibrous web on the second fibrous web wherein the first fibrous web forms a first side of the composite fibrous web and the second fibrous web forms a second side of the composite fibrous web; and (c) subjecting the composite fibrous web to bonding/integrating treatment to bind or integrate at least part of fibers constituting the first fibrous web and the second fibrous web to obtain a nonwoven.

As another example, the nonwoven of the present invention may be manufactured in a continuous process. For example, the process may comprise the steps of (a) forming a first fibrous web comprising first fibers and overlaying a second fibrous web comprising second fibers on top of the first fibrous web to form a composite fibrous web; and (b) subjecting the complex fibrous web to bond treatment to bind or integrate at least part of fibers constituting the first fibrous web and the second fibrous web to obtain a nonwoven.

The first fibrous web and the second fibrous web may be carded webs such as parallel webs, semi-random webs, random webs, cross-webs, criss-cross webs, and the like, air-laid webs, wet-laid webs, and spunbond webs, and the like.

The bonding treatment of a composite fibrous web can be conducted using any conventionally known fiber bonding method. Examples of such a bonding method include hot air through-type thermal bonding, ultrasonic bonding, and an infrared thermal treatment apparatus.

In air-through bonding process, the fibers are bonded by heating energy provided by hot air through the fibrous web(s). The hot air applied to the fibrous web(s) generates pressure on the web which results in web caliper reduction. In production of the nonwoven laminate of the present invention, the air flow pressure may be controlled in a relative low range compared to traditional air-through process. If the air flow pressure is too high, both fibrous webs may be compressed and densified which may not bring the desirable pore size gradient structure. If the air flow pressure is too low, it may negatively impact on fiber bonding strength and eventually on nonwoven strength, and may cause a fuzz issue on surfaces of a finished nonwoven. In production of the nonwoven laminate of the present invention, the air flow pressure may be controlled in a relative low range compared to traditional air-through process. An appropriate air flow pressure may be decided considering fiber deniers of constituting fibers, a basis weight of each fibrous web, a total basis weight of fibrous webs, and a basis weight ratio of fibrous webs, etc.

### Application of Nonwoven

The nonwoven of the present has a pore size gradient in such a way that a mean pore size in an upper layer is greater than a mean pore size in a lower layer, so that the lower layer has high capillary suction power than the upper layer which creates a capillary capability to drain fluid from the first layer to the second layer. In addition, the first layer comprises first fibers having a fiber fineness equal to or smaller than a fiber fineness of the second fiber constituting the second layer, the nonwoven still can have a soft and smooth first layer surface. As such, the nonwoven of the present invention can be used in applications in which the nonwoven is in contact with the skin, specifically applications in which the first layer is the surface that is in contact with the skin. For example, the nonwoven of the present invention can be used in applications such as products that contact human or non-human animal skin, such as various absorbent articles; face masks, base fabric of cooling/heating pads and similar cosmetic/medical-use patches, wound surface protection sheets, nonwoven bandages, hemorrhoid pads, warming devices that directly contact the skin (e.g. disposable hand warmers), base fabric of various animal-use patches, and similar skin covering sheets; makeup removal sheets, anti-perspirant sheets, bottom wipes and similar wipes for use on a person, various wiping sheets for use on animals, and the like. The nonwoven of the present invention can be used in applications in which the nonwoven handles fluid transfer.

The nonwoven of the present invention may be used as a topsheet for an absorbent article in which the surface of first layer is in contact with the skin.

The nonwoven of the present invention may be also used as a fluid management layer for an absorbent article in which the first layer is towards a topsheet of the absorbent article.

The nonwoven of the present invention may be also used as an outer most layer forming an outer surface such as an outer cover and an outer belt layer of the absorbent article.

### Absorbent Article

An absorbent article according to the present invention comprises a wearer-facing surface, a garment-facing surface, a liquid permeable topsheet, a liquid impermeable backsheet, an absorbent core disposed between the topsheet and the backsheet, and optionally a fluid management layer disposed between the topsheet and the absorbent core, wherein at least one of the topsheet and the optional fluid management layer comprises the nonwoven of the present invention in such a way that the first layer towards the wearer-facing surface.

An absorbent article according to the present invention comprises a wearer-facing surface, a garment-facing surface, a liquid permeable topsheet, a liquid impermeable backsheet, an absorbent core disposed between the topsheet and the backsheet, and an outermost layer forming the garment-facing surface wherein the outermost layer comprises the nonwoven of the present invention in such a way that the first layer towards the garment-facing.

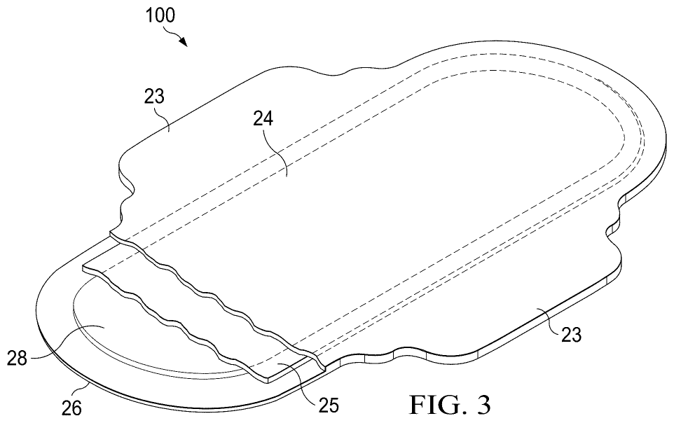

Absorbent articles will now be generally discussed and further illustrated in the form of a sanitary napkin 100 as exemplarily represented in FIG. 3 which is a plan view of the exemplary sanitary napkin 100 in a flattened-out configuration and the wearer facing side turned up.

Referring to FIG. 3, an absorbent article 100, a sanitary napkin 100 for example, comprises a topsheet 24 having a wearer facing surface and a garment facing surface positioned opposite to the wearer facing surface. The absorbent article further comprises a backsheet 26 having a garment facing surface and a wearer facing surface positioned oppositely to the garment facing surface, the backsheet 26 being at least partially joined to the topsheet 24. The absorbent article also comprises an absorbent core 28 positioned between the topsheet 24 and the backsheet 26. The absorbent article may further comprise an optional fluid management layer 25 positioned between the topsheet 24 and the absorbent core 28. The absorbent article may further comprise a pair of flaps or wings 23. The topsheet 24, the backsheet 26, the fluid management layer 25, and the absorbent core 28, and other optional elements can be assembled in a variety of well-known configurations.

The backsheet 26 and the topsheet 24 can be secured together in a variety of ways. The topsheet 24 and the backsheet 26 can be joined to each other by using an adhesive, heat bonding, pressure bonding, ultrasonic bonding, dynamic mechanical bonding, or a crimp seal. A fluid impermeable crimp seal can resist lateral migration (“wicking”) of fluid through the edges of the product, inhibiting side soiling of the user's undergarments.

Absorbent articles comprising the nonwoven of the present disclosure may be made by any suitable methods known in the art. The absorbent articles of the present invention may be produced industrially by any suitable means. The different layers may thus be assembled using standard means such as embossing, thermal bonding, gluing or any combination thereof.

### Topsheet

A topsheet is generally liquid permeable and is configured to receive fluids being excreted from the body and aid in directing the fluids toward an optional fluid management layer and/or the absorbent core. One of the important qualities of a topsheet is the ability to reduce ponding of the fluids on the surface of topsheet before the fluids are able to be absorbed by a layer or the absorbent core underneath the topsheet. Another desirable quality of a topsheet is to reduce rewet of the topsheet. It is also desirable that the topsheet is to present a soft feel to the wearer's skin.

The topsheet may be joined to portions of the backsheet, the absorbent core, and/or any other layers as is known to those of ordinary skill in the art.

A suitable topsheet can be made of various materials such as woven and nonwoven materials. As an option, at least a portion of the topsheet can be rendered hydrophilic, by the use of any known method for making topsheets containing hydrophilic components.

When a topsheet comprises the nonwoven of the present invention, the nonwoven is disposed such a way that the first layer is towards the wearer-facing surface.

### Fluid Management Layer

The absorbent article of the present invention may comprise a fluid management layer. One function of a fluid management layer is to quickly acquire liquids or other bodily exudates from a topsheet and transfer and distribute them to the absorbent core in an efficient manner.

A fluid management layer in the absorbent article of the present invention is disposed directly or indirectly on top of an absorbent core.

### Backsheet

Any conventional liquid impervious backsheet materials commonly used for absorbent articles may be used as backsheet. In some embodiments, the backsheet may be impervious to malodorous gases generated by absorbed bodily discharges, so that the malodors do not escape. The backsheet may or may not be breathable.

### Absorbent Core

It may be desirable that the absorbent article further comprises an absorbent core disposed between the topsheet and the backsheet.

The absorbent core comprises an absorbent material. The absorbent material in the absorbent core can be any liquid-absorbent material commonly used in disposable absorbent articles such as comminuted wood pulp, which is generally referred to as airfelt or fluff. Examples of other suitable liquid-absorbent materials include creped cellulose wadding; melt blown polymers, including co-form; chemically stiffened, modified or cross-linked cellulosic fibers; tissue, including tissue wraps and tissue laminates, absorbent foams, absorbent sponges, superabsorbent polymers (herein abbreviated as “SAP”), absorbent gelling materials, or any other known absorbent material or combinations of materials. The term “superabsorbent polymer” refers herein to absorbent material, which may be cross-linked polymer, and that can typically absorb at least 10 times their weight of an aqueous 0.9% saline solution as measured using the Centrifuge Retention Capacity (CRC) test (EDANA method WSP 241.2-05E). The SAP may in particular have a CRC value of more than 20 g/g, or more than 24 g/g, or of from 20 to 50 g/g, or from 20 to 40 g/g, or from 24 to 30 g/g. The SAP may be typically in particulate forms (superabsorbent polymer particles), but it not excluded that other forms of SAP may be used such as a superabsorbent polymer foam, for example.

### TEST METHODS

All measurements are performed in a laboratory maintained at 23° C.±2° C. and 50%±2% relative humidity and test samples are conditioned in this environment for at least 2 hours prior to testing unless differently specified.

### 1. Fiber Property Test

### 1.1 Sample Preparation

When a fiber is available in a raw material form, at least a 30 mm length of fiber is cut from the raw material. When a fiber is a component of a nonwoven, the fiber is removed from the nonwoven to make sure that the minimum length of a single fiber is 30 mm.

### 1.2 Fiber Elasticity Rate and Fiber Recovery

The fiber elasticity rate is measured according to GB/T14338-2008 Testing Method for Crimping Performance Of Man-Made Staple Fibers. If it is a filament fiber, the fiber is cut into staple fibers with a length of 30-60 mm.

Randomly select 20 fibers from the conditioned samples, placed on a velvet board. Clamp a fiber with a tension clamp and hang it on the force measuring hook of the crimping elasticity tester. Then use tweezers to place the other end of the fiber in the lower gripper, apply a light load, and record the length L0; then apply a heavy load and record the length L1; Remove the heavy load, wait for 2 mins, measure the fiber length L2 under light load.

$$Fiber elasticity rate {J}_{d} = ( L 1 - L 2 ) / ( L 1 - L 0 ) * 100 \%$$

The higher the elasticity rate, the better the fiber elasticity performance.

$$Fiber recovery Jr = ( L 2 - L 0 ) / L 0 * 100 \% ,$$

The lower the value, the better the fiber recovery.

### 2. Fiber Decitex Measurement

Textile webs (e.g., woven, nonwoven, airlaid) are comprised of individual fibers of material. Fibers are measured in terms of linear mass density reported in units of decitex (Dtex). The decitex value is the mass in grams of a fiber present in 10,000 meters of that fiber. The decitex value of the fibers within a web of material is often reported by manufacturers as part of a specification. If the decitex value of the fiber is not known, it can be calculated by measuring the cross-sectional area of the fiber via a suitable microscopy technique such as scanning electron microscopy (SEM), determining the composition of the fiber with suitable techniques such as FT-IR (Fourier Transform Infrared) spectroscopy and/or DSC (Dynamic Scanning Calorimetry), and then using a literature value for density of the composition to calculate the mass in grams of the fiber present in 10,000 meters of the fiber. All testing is performed in a room maintained at a temperature of 23° C.±2.0° C. and a relative humidity of 50%±2% and samples are conditioned under the same environmental conditions for at least 2 hours prior to testing.

If necessary, a representative sample of web material of interest can be excised from an absorbent article. In this case, the web material is removed so as not to stretch, distort, or contaminate the sample.

SEM images are obtained and analyzed as follows to determine the cross-sectional area of a fiber. To analyze the cross section of a sample of web material, a test specimen is prepared as follows. Cut a specimen from the web that is about 1.5 cm (height) by 2.5 cm (length) and free from folds or wrinkles. Submerge the specimen in liquid nitrogen and fracture an edge along the specimen's length with a razor blade (VWR Single Edge Industrial Razor blade No. 9, surgical carbon steel). Sputter coat the specimen with gold and then adhere it to an SEM mount using double-sided conductive tape (Cu, 3M available from electron microscopy sciences). The specimen is oriented such that the cross section is as perpendicular as possible to the detector to minimize any oblique distortion in the measured cross sections. An SEM image is obtained at a resolution sufficient to clearly elucidate the cross sections of the fibers present in the specimen. Fiber cross sections may vary in shape, and some fibers may consist of a plurality of individual filaments. Regardless, the area of each of the fiber cross sections is determined (for example, using diameters for round fibers, major and minor axes for elliptical fibers, and image analysis for more complicated shapes). If fiber cross sections indicate inhomogeneous cross-sectional composition, the area of each recognizable component is recorded and tax contributions are calculated for each component and subsequently summed. For example, if the fiber is bi-component, the cross-sectional area is measured separately for the core and sheath, and date contribution from core and sheath are each calculated and summed. If the fiber is hollow, the cross-sectional area excludes the inner portion of the fiber comprised of air, which does not appreciably contribute to fiber tax. Altogether, at least 100 such measurements of cross-sectional area are made for each fiber type present in the specimen, and the arithmetic mean of the cross-sectional area aₛₖ for each are recorded in units of micrometers squared (μm²) to the nearest 0.1 μm².

Fiber composition is determined using common characterization techniques such as FTIR spectroscopy. For more complicated fiber compositions (such as polypropylene core/polyethylene sheath bi-component fibers), a combination of common techniques (e.g., FTIR spectroscopy and DSC) may be required to fully characterize the fiber composition. Repeat this process for each fiber type present in the web material.

The decitex dₖ value for each fiber type in the web material is calculated as follows:

$${d}_{k} = 10000 m \times {a}_{k} \times {ρ}_{k} \times 1 {0}^{- 6}$$

where dₖ is in units of grams (per calculated 10,000 meter length), Aₖ is in units of μm², and ρₖ is in units of grams per cubic centimeter (g/cm³). Decitex is reported to the nearest 0.1 g (per calculated 10,000 meter length) along with the fiber type (e.g., PP, PET, cellulose, PP/PET bico).

### 3. Basis Weight of Nonwoven

The basis weight of nonwoven substrates are measured according to “ISO 9073-1:1989 Textiles—Test methods for nonwovens—Part 1: Determination of mass per unit area”.

Measurements are made on test samples taken from rolls or sheets of the raw material, or test samples obtained from a material layer removed from an absorbent article. When excising the material layer from an absorbent article, use care to not impart any contamination or distortion to the layer during the process.

### 4. Nonwoven Cross Section Images

If a nonwoven is available in its raw material form, a specimen with the size about 25 mm×25 mm or a bigger size is cut from the raw material to include. If a nonwoven is a component layer such as a topsheet of an absorbent article, the absorbent article this size is cut into a size about 25 mm×25 mm or a bigger size, and the nonwoven layer is removed from the absorbent article, using a razor blade to excise the nonwoven layer from the underling layers of the absorbent article. A cryogenic spray (such as Cyto-Freeze, Control Company, Houston TX) or other suitable solvents that do not permanently alter the properties of the nonwoven layer composition may be used to remove the nonwoven layer specimen from the underling layers if necessary. Any remaining adhesive may be removed from the specimen by the following steps using Tetrahydrofuran (THF) as solvent.

The nonwoven specimen is placed on the microscope stage using double-sided conductive tape or clapped to fix the specimen. An appropriate magnification can be chosen such that features in the nonwoven specimen are suitably clear and enlarged for measurement.

Microscopic images of nonwoven specimens are taken by an optical microscope, 3CCD optical microscopy-Keyence VHX5000 or equivalent, and are used to measure an upper portion height and a lower portion height of nonwoven.

### 5. Pore Size Measurement

The pore size of a fibrous structure sample is measured using a micro-CT imaging and analysis method. It is based on analysis of a 3D x-ray sample image obtained on a micro-CT instrument (a suitable instrument is the Samco μCT 50 available from sconce Medical AG, Switzerland, or equivalent). The micro-CT instrument is a cone beam micro-tomograph with a shielded cabinet. A maintenance free x-ray tube is used as the source with an adjustable diameter focal spot. The x-ray beam passes through the sample, where some of the x-rays are attenuated by the sample. The extent of attenuation correlates to the mass of material the x-rays have to pass through. The transmitted x-rays continue on to the digital detector array and generate a 2D projection image of the sample. A 3D image of the sample is generated by collecting several individual projection images of the sample as it is rotated, which are then reconstructed into a single 3D image. The instrument is interfaced with a computer running software to control the image acquisition and save the raw data. The 3D image is then analyzed using image analysis software (a suitable image analysis software is MATLAB available from The Mathworks, Inc., Natick, MA, or equivalent) to measure the desired properties of regions within the sample.

### 5.1 Sample Preparation

The test sample is prepared as follows. A single sheet of the test material is placed onto a rigid, horizontal benchtop and a sharp die cutter is used to punch out a circular sample that has a diameter of 7 mm. The test sample is obtained from an area on the test material that is free of folds or wrinkles, and care is used to prevent any contamination or distortion to the test sample during the preparation process. Additional test samples from separate regions within a given test material can be prepared for analysis and comparison. The test samples are conditioned at about 23° C.±2° C. and about 50%±2% relative humidity for 2 hours prior to testing.

### 5.2 Image Acquisition

The micro-CT instrument is set up and calibrated according to the manufacturer's specifications. The test sample is placed into the appropriate holder, between two disks of low-density material which have a diameter of 7 mm. The test sample is scanned under a compressive load by adding a weight to the uppermost low-density disk, with the mass sufficient to apply a pressure of 2 kPa over the 7 mm diameter test sample. Once the compressive load has been applied, the weight is clamped in place to prevent movement during the scan.

The 3D image field of view is approximately 10 mm on each side in the x-y plane with a resolution of approximately 5124 by 5124 pixel, and with a sufficient number of 2 micron thick slices collected to fully include the entire z-direction of the test sample. The reconstructed 3D image contains isotropic voxel of 2 microns. Images are acquired with a source at 45 kVp and 88 μA with no additional low energy filter. The current and voltage setting should be optimized to produce the maximum contrast in the projection data with sufficient x-ray penetration through the test sample, but once optimized, the settings are held constant for all subsequent test sample replicates. A total of 1800 projection images are obtained with an integration time of 750 ms and 6 averages. The projection images are reconstructed into the 3D image and saved in 16-bit format to preserve the full detector output signal for analysis. A file of the resulting data set is of a proprietary format according to the instrument supplier's instruction, and is referred to as the ISQ file in the following image visualization and analysis steps.

### 5.3 Image Visualization and Analysis

The objective of the image analysis is to measure a 3-dimensional void cell diameter in the first layer and the second layer of a fibrous structure sample that consists of a topsheet (first layer) and a secondary topsheet (second layer). The ISQ files described above, are read into high end image visualization and analysis platform, for example, Avizo 9.2.0 (FEI, Houston, Tex., USA). Upon inspection of obtained visualized 3-dimensional data, 3 different regions in each of the two layers, that is a topsheet (first layer), and a secondary topsheet (second layer), are analzyed and compared. For each fibrous structure sample there is therefore 6 subvolumes chosen for measurements of 3-dimensional void size diameter. To make measurements of Porosity and 3D void cell size distribution, the following steps are performed:
1) An automated thresholding algorithm practicing Otsu's method (A Threshold Selection Method from Gray-Level Histograms”, Nobuyuki Otsu, 2EEE Transactions On Systems Man, and Cybernetics, VOL. SMC-9, NO. 1, January 1979) was applied to each of the datasets resulting in a binary image (0-1) representing the fibers (1) and void space (0).
2) A void cell diameter is measured according to the method disclosed in a paper published by Tor Hildebrand (T. Hildebrand and P. Ruegsegger, “A new method for the model-independent assessment of thickness in three-dimensional images. Journal of Microscopy, 185:67-75, 1996). First, the void space is then fitted with spheres of different sizes, where larger spheres cover up smaller spheres using a software working the method disclosed in the paper, for example, IPL software from Scanco Medical, Zurich, Switzerland). This final tessellation of the void space provides a distribution of spheres that completely cover the void space. The volume weighted mean diameter represents the mean void cell diameter. This is implemented through an image analysis platform, for example Matlab R2016B, (Natick, Mass., USA) as module in Avizo 9.2.0.
3) The resulting measurements are brought into Excel 2013. The values of volume weighted mean diameter of the three regions of each layer are then averaged to produce a single value void cell diameter for that layer. The void cell diameter of the region is a mean pore size of the layer and is reported as Mean Pore Size to the nearest micron.

### 6. Caliper Recovery Factor

6.1 Sample Preparation

If a nonwoven is available in its raw material form, a specimen with the size about 25 mm×25 mm or a bigger size is cut from the raw material. If a nonwoven is a component layer such as a topsheet of an absorbent article, the absorbent article this size is cut and the nonwoven layer is removed from the absorbent article, using a razor blade to excise the nonwoven layer from the underling layers of the absorbent article. A cryogenic spray (such as Cyto-Freeze, Control Company, Houston TX) or other suitable solvents that do not permanently alter the properties of the nonwoven layer composition may be used to remove the nonwoven layer specimen from the underling layers if necessary. Any remaining adhesive may be removed from the specimen by the following steps using Tetrahydrofuran (THF) as solvent.
1) In a hood, transfer 1 liter of THF into the 3-4 liter beaker.
2) Submerge specimen in the 1 liter of THF.
3) Place beaker on shaking table and stir gently for 15 minutes and keep solution with sample sit for 5 additional minutes.
4) Take specimen out of THF solution, and carefully squeeze THE solution out of specimen.
5) Let specimen air dry in hood for a minimum of 15 minutes.

To obtain a nonwoven cross section specimen, the nonwoven is laid on a flat bench with a first side upward.

### 6.2 Image Generation

Microscopic images of nonwoven specimens are taken by an optical microscope, 3CCD optical microscopy-Keyence VHX5000 or equivalent, and are used to measure the height of nonwoven.

The nonwoven specimen is placed on the microscope stage using double-sided conductive tape or clapped to fix the specimen. An appropriate magnification can be chosen such that features in the nonwoven specimen are suitably clear and enlarged for measurement.

### 6.3 Nonwoven Caliper Measurement

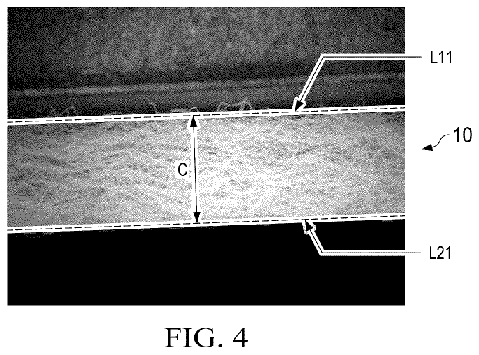

A flat area having no deformation in a sample nonwoven 10 is carefully selected to measure a nonwoven caliper. Referring to FIG. 4, draw a line L21 contacting the bottom surface of nonwoven 10. Draw another line L11 contacting the upper surface of nonwoven 10 in in parallel to line L21. A distance between line L11 and line L21 is measured and reported as a nonwoven caliper C. A caliper to the nearest 0.01 mm is reported. For each nonwoven sample, 3 images of different nonwoven cross section parts are tested. The reported value is the average of the 3 recorded measurements for each nonwoven.

### 6.4 Reloft of Nonwoven

A sample nonwoven is placed in an oven set an oven temperature of 55° C. for 5 minutes for relofting, and removed from the oven and placed in a room temperature.

### 6.5 Caliper Recovery Factor

$$The caliper recovery factor , as mentioned previously , is the caliper recovery per 10 gsm of basis weight of the sample . So , the equation is \{ ( ( Reloft caliper - original caliper ) / original caliper ) \times 100 \} / ( basis weight / 10 ) .$$

At least 10 replica of a sample are tested and an average value (arithmetic mean) of the tested replica is reported as caliper recovery factor.

### EXAMPLES

### Example 1: Fiber Properties

Fiber properties of various fibers were measured according to Fiber Property Test, and results are listed in Table 1. All Tested fibers were core/sheath (50:50) bicomponent fibers.

TABLE 1

| | Fiber 1 | Fiber 2 | Fiber 3 | Fiber 4 | Fiber 5 |
|---|---|---|---|---|---|
| Resin | 2.5D PE/PTT | 2.3D PE/PBT | 2D PE/PET | 2D PE/PP | 2.5 PE/PET |
| | | | | | eccentric |
| Fiber elasticity | 90.14 | 90.01 | 74.44 | 75.26 | 84.26 |
| rate (%) | | | | | |
| Fiber recovery | 2.27 | 4.15 | 10.04 | 6.67 | 7.44 |
| (%) | | | | | |

### Example 2: Nonwoven Preparation

Nonwovens 1-4 were produced using various first fibrous webs and second fibrous webs indicated in Table 2. For each of Nonwovens 1-4, a composite nonwoven web was fabricated using parallel carding machines by laying a first fibrous web on a conveyor belt and overlaying a second fibrous web on the first fibrous web. Each composite nonwoven web was heat-treated at the temperatures 130-140° C. using a hot air through-type thermal treatment apparatus using a conventional process to produce a nonwoven. All fibers for Nonwovens 1-4 are core/sheath type bicomponent fibers.

TABLE 2

| | Nonwoven 1 | Nonwoven 2 | Nonwoven 3 | Nonwoven 4 |
|---|---|---|---|---|
| Basis weight (gsm) | 40 | 40 | 21 | 40 |
| First layer | 25 gsm | 25 gsm | 8 gsm | 25 gsm |
| | 2.5D PE/PTT | 2.3D PE/PBT | 2.0D PE/PET | 2.5D PE/PET |
| Second layer | 15 gsm | 15 gsm | 13 gsm | 15 gsm |
| | 3.5D PE/PET | 3.5D PE/PET | 4.0D PE/PP | 2.0D PE/PP |

### Example 3. Nonwoven Properties

The obtained nonwovens were evaluated as described below.

Cross-section images of nonwovens were taken according to Nonwoven Cross Section Images disclosed herein under magnification of 50×.

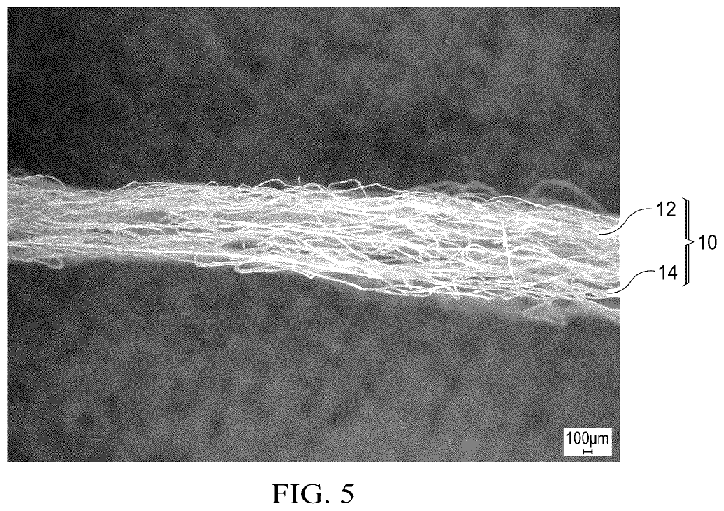

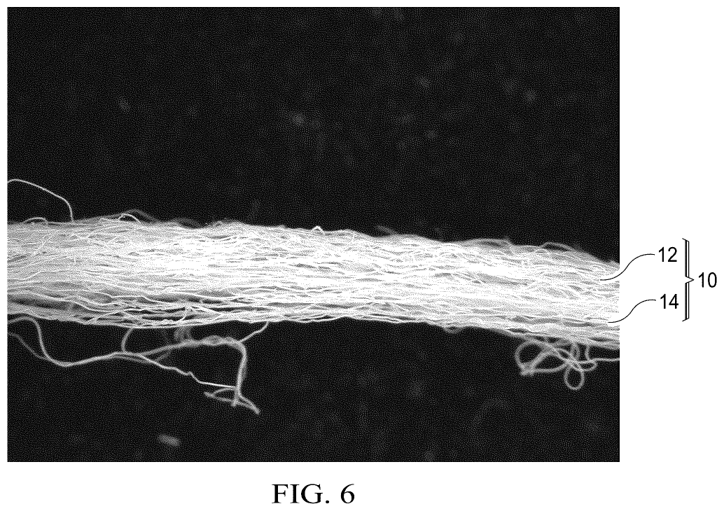

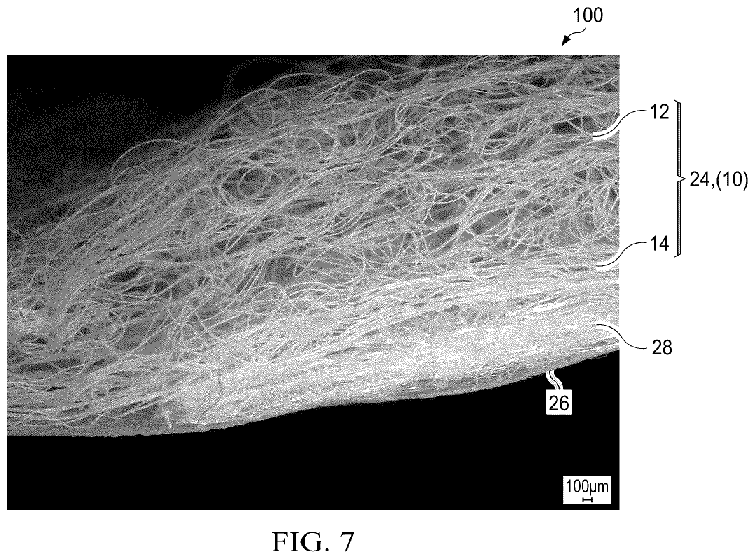

FIG. 1A is a microscopic image of a cross-section view of Nonwoven 1. FIG. 1B is a microscopic image of a cross section view of Nonwoven 1 after reloft according to 6.4 Reloft of nonwoven. FIGS. 2, 5 and 6 are microscopic images of cross section views of Nonwovens 2, 3 and 4, respectively. FIG. 7 is a microscopic image of a partial cross section view of an absorbent article 100 comprising a topsheet 24 made by Nonwoven 1 (nonwoven 10), an absorbent core 28 and backsheet 26. It was observed the first layer 12 has pores significantly bigger than pores in the second layer 14.

Mean pore sizes and calipers of nonwovens were measured according to Pore Size Measurement and Caliper Recovery Factor and are indicated in Table 3.

TABLE 3

| | | Nonwoven 1 | Nonwoven 2 | Nonwoven 3 | Nonwoven 4 |
|---|---|---|---|---|---|
| Mean pore | First layer | 147 | 160 | 99 | 105 |
| size (μm) | Second | 139 | 124 | 128 | 77 |
| | layer | | | | |

| | | | | |
|---|---|---|---|---|
| Caliper Recovery Factor | 7.65 | 10.22 | 0.07 | 1.13 |
| Caliper- initial | 3355 | 2772 | 1372 | 2117 |
| Caliper- reloft | 4381 | 3905 | 1374 | 2213 |

Nonwovens 1 and 2 of the present invention have a bigger mean pore size in first layer 12 than in the second layer 14 despite the first fiber constituting the first layer 12 has a smaller fiber fineness than the second fiber constituting the second layer 14.

Nonwoven 3 as a comparison has a first layer 12 comprising first fibers and a second layer 14 comprising second fibers, the second fiber having a fiber fine greater than a fiber fineness of the first fibers fineness. In Nonwoven 3, the first layer 12 has a smaller mean pore size than the second layer 14 as anticipated.

Nonwoven 4, another comparison, has a first layer 12 comprising first fibers and a second layer 14 comprising second fibers, the second fiber having a fiber fine smaller than a fiber fineness of the first fibers fineness. In Nonwoven 4, the first layer 12 has a greater mean pore size than the second layer 14 as anticipated.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A nonwoven comprising:
a first layer comprising first fibers and having a first mean pore size as measured by Pore Size Measurement, the first fiber having a first fiber fineness as measured by Fiber Decitex Measurement; and
a second layer comprising second fibers and having a second mean pore size as measured by Pore Size Measurement, the second fiber having a second fiber fineness as measured by Fiber Decitex Measurement,
wherein the second fiber fineness is equal to or greater than the first fiber fineness, and
wherein the second mean pore size is smaller than the first mean pore size.

2. The nonwoven of claim 1, wherein the nonwoven comprises a caliper recovery factor no less than about 5% as measured according to Recovery Factor.

3. The nonwoven of claim 1, wherein the first fiber comprising a polymer selected from the group consisting of comprising polytrimethylene terephthalate (PTT), polybutylene terephthalate (PBT) and a combination thereof.

4. The nonwoven of claim 1, wherein the first layer comprises the first fiber at least 30% of total fibers constituting the first layer.

5. The nonwoven of claim 1, wherein the first fiber comprises a fiber recovery no greater than about 6% as measured according to Fiber Property Test.

6. The nonwoven of claim 1, wherein the first fiber comprises a fiber elasticity rate no less than about 85% as measured according to Fiber Property Test.

7. The nonwoven of claim 1, wherein the second fiber comprises a thermoplastic fiber.

8. The nonwoven of claim 1, wherein the first fiber has a fiber thickness in the range from about 0.8 denier to about 6 denier.

9. The nonwoven of claim 1, wherein the second fiber has a fiber thickness in the range from about 1.5 denier to about 6 denier.

10. The nonwoven of claim 1, wherein the second fiber differs from the first fiber.

11. The nonwoven of claim 1, wherein the nonwoven has a basis weight of from about 15 g/m² to about 100 g/m².

12. The nonwoven of claim 1, wherein a ratio of a basis weight of the first layer to the second layer is from about 80/20 to about 20/80.

13. The nonwoven of claim 1, wherein the nonwoven is an air through bonded nonwoven, a spunlace nonwoven, a needle punch nonwoven, a calendar bonded nonwoven or a resin bonded nonwoven.

14. A nonwoven comprising:
a first layer comprising first fibers, the first fiber comprising a polymer selected from the group consisting of comprising polytrimethylene terephthalate (PTT), polybutylene terephthalate (PBT) and a combination thereof; and
a second layer comprising second fibers, the second fiber comprises a thermoplastic fiber.

15. The nonwoven of claim 14, wherein the second fibers differs from the first fibers.

16. The nonwoven of claim 14, wherein the first fibers has a fiber thickness in the range from about 0.8 denier to about 6 denier.

17. The nonwoven of claim 14, wherein the second fibers has a fiber thickness in the range from about 1.5 denier to about 6 denier.

18. An absorbent article having a wearer-facing surface and a garment-facing surface, the absorbent article comprising:
a topsheet, a backsheet, an absorbent core disposed between the topsheet and the backsheet, and a fluid management layer disposed between the topsheet and the absorbent core,
wherein at least one of the topsheet and the fluid management layer comprises a nonwoven, wherein the nonwoven comprises:
a first layer comprising first fibers, the first fiber comprising a polymer selected from the group consisting of comprising polytrimethylene terephthalate (PTT), polybutylene terephthalate (PBT) and a combination thereof; and
a second layer comprising second fibers, the second fiber comprises a thermoplastic fiber, and
wherein the first layer towards the wearer-facing surface.

19. The absorbent article of claim 18, wherein the nonwoven has a basis weight of from about 15 g/m² to about 100 g/m².

20. The absorbent article of claim 18, wherein a ratio of a basis weight of the first layer to the second layer is from about 80/20 to about 20/80.
